(19) United States
(12) Patent Application Publication
(10) Pub. No.: US 2022/0134072 A1
(43) Pub. Date: May 5, 2022

(54) MINIATURIZED DELIVERY SYSTEM AND METHOD

(71) Applicant: KING ABDULLAH UNIVERSITY OF SCIENCE AND TECHNOLOGY, Thuwal (SA)

(72) Inventors: Jürgen KOSEL, Thuwal (SA); Khalil MOUSSI, Thuwal (SA)

(21) Appl. No.: 17/437,572

(22) PCT Filed: Mar. 17, 2020
(86) PCT No.: PCT/IB2020/052434

§ 371 Date: Sep. 9, 2021

Related U.S. Application Data
(60) Provisional application No. 62/820,542, filed on Mar. 19, 2019.

(51) Int. Cl. A61M 37/00; A61M 5/20

(52) U.S. Cl. CPC A61M 37/0015; A61M 5/2046; A61M 2037/0061; A61M 2037/003; A61M 2037/0023

(57) ABSTRACT

A miniature delivery system includes a base; a pumping mechanism attached to the base; and a housing having needles, the housing being attached to the base so that the pumping mechanism is enclosed by the housing. The needles are configured to not buckle or break when pressed directly into a skin or organ of a human to which the miniature delivery system is attached to, and the pumping mechanism is configured to pump a fluid from the housing into the skin or organ, through the needles.

### CROSS-REFERENCE TO RELATED APPLICATIONS

This application claims priority to U.S. Provisional Patent Application No. 62/820,542, filed on Mar. 19, 2019, entitled “MINIATURIZED DELIVERY SYSTEM,” the disclosure of which is incorporated herein by reference in its entirety.

### BACKGROUND

### Technical Field

Embodiments of the subject matter disclosed herein generally relate to a system and method for delivering a drug, and more particularly, to a miniaturized drug delivery system that is suitable for in-vivo biomedical applications.

### Discussion of the Background

Conventional drug delivery routes provide limited control over the spatial and temporal resolution of the drug release. Often, the desired availability of the therapeutic drug in the target site can only be achieved by either increasing the dose volume or the dosing frequency, both of which are undesired, due to side effects and low patient compliance. One way to circumvent this issue is via direct injection of the drugs into the target site. This strategy, however, cannot be used to reach remote areas of the body and has to be done repeatedly to achieve the desired therapeutic effect, leading to trauma and risk of infections. As such, alternative approaches to drug administration have been intensely investigated.

One such approach is the use of coatings that confer selectivity to a generic drug. The drug can be coated with polymers, nanoparticles, liposome, or specific cell-receptor ligands that allow the drug to evade being systematically cleared out by the body and accumulate at the desired target area. The drug cargo may then be released using an external stimulus, such as localized heating, or environment sensing mechanisms, such as pH-sensitive hydrolyzing polymers. Microparticles or nanoparticles can be exploited similarly to carry and release drugs. While this approach may allow for selective targeting, it offers little or no control over the release rate of the drug.

These issues have fueled interest in the use of biocompatible and miniaturized delivery platforms that can be implanted using minimally invasive procedures. Such platforms allow for a controlled and targeted release of the drugs by using actuators that are coupled to a drug reservoir. Osmotic actuators have been very popular, but provide no or limited control of the delivery rate [1]. Electrolytic actuators have been gaining traction being implemented into drug delivery platforms, due to their simplicity and efficiency [2].

Conventional electrolytic actuators utilize a diaphragm-design, in which the electrolysis of water drives the deflection of the diaphragm. This deflection pushes the drug from an adjacent reservoir compartment through a funneled cannula to the target site. In this configuration, the release rate can be controlled by limiting the supplied current driving the electrolysis reaction. Versatile delivery systems with attractive features including wireless operation and valve control have been developed, but integration into a compact package is lacking [3].

Thus, there is a need for a new system that is capable of delivering the drug directly to the target, that can control the amount and rate of the drug being delivered to the target, is small enough to fit on the target, and is also biocompatible with the target.

### BRIEF SUMMARY OF THE INVENTION

According to an embodiment, there is a miniature delivery system that includes a base, a pumping mechanism attached to the base, and a housing having needles, the housing being attached to the base so that the pumping mechanism is enclosed by the housing. The needles are configured to not buckle or break when pressed directly into a skin or organ of a human to which the miniature delivery system is attached to. The pumping mechanism is configured to pump a fluid from the housing into the skin or organ, through the needles.

According to another embodiment, there is a miniature delivery kit for delivering a drug, the kit including a delivery system and means for attaching the delivery system to a skin or organ. The delivery system includes a base, a bellows membrane directly attached to the base, and a housing having needles, the housing being attached to the base so that the bellows membrane is enclosed by the housing. The bellows membrane moves from a retracted state, in which an external face is farthest from an internal face of the housing, to an extended state, in which the external face is closest to the internal face of the housing. The external face of the bellow membrane is substantially parallel to the internal face of the housing in both the retracted state and the extended state.

According to still another embodiment, there is a method for delivering a drug to a skin or organ of a human. The method includes loading a delivery system with the drug; attaching the delivery system directly to the skin or organ by pushing one or more needles directly into the skin or organ, wherein the one or more needles are part of a housing of the delivery system; actuating a bellows membrane of the delivery system to move from a retracted state to an extended state; delivering the drug through the one or more needles to the skin or organ; and removing a power supply from the delivery system to stop the drug delivery.

### BRIEF DESCRIPTION OF THE DRAWINGS

For a more complete understanding of the present invention, reference is now made to the following descriptions taken in conjunction with the accompanying drawings, in which:

FIG. 1 illustrates a miniaturized delivery system configured to be attached directly to a skin or organ of a person;

FIG. 2 illustrates the miniaturized delivery system having a bellows membrane in a retracted state;

FIG. 3 illustrates the miniaturized delivery system having a bellows membrane in an extended state;

FIG. 4A is an overview of the miniaturized delivery system and its power supply mechanism and FIG. 4B is an overview of an array of miniaturized delivery systems;

FIGS. 5A to 5D illustrate various miniaturized delivery systems having different microneedles;

FIG. 6 illustrates the details of the various miniaturized delivery systems shown in FIGS. 5A to 5D;

FIGS. 7A to 7D illustrate the flow rate versus the pressure flow through the needles of the miniaturized delivery systems illustrated in FIGS. 5A to 5D;

FIG. 8 illustrates the force applied to the microneedles versus the outer diameter of the microneedles;

FIG. 9 illustrates the cell viability when in contact with the miniaturized delivery system;

FIGS. 10A to 10C illustrate the fabrication process for the substrate and the electrodes of the miniaturized delivery system;

FIG. 11 illustrates the various steps for forming the bellows membrane of the miniaturized delivery system;

FIG. 12 shows a cross-section through the bellows membrane and various parameters of the membrane;

FIG. 13 illustrates how the housing and the bellows membrane of the miniaturized delivery system are attached to the substrate;

FIG. 14 illustrates how the miniaturized delivery system can be attached to the skin;

FIG. 15 illustrates how the miniaturized delivery system can be attached to an organ; and

FIG. 16 is a flowchart of a method for delivering a drug to a person with a miniaturized delivery system.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to an implantable 3D printed drug delivery system that is directly attached to an organ of the human body, and delivers a desired amount of a drug directly to the organ. However, the embodiments to be discussed next are not limited to a 3D printed system, or a drug delivery system, but may be applied to other delivery systems.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel drug delivery system includes an electrolytic pump driving a micro bellows membrane as an actuator for delivery of the drug through microneedles directly formed on a housing of the system. A two-photon polymerization 3D printing technique is used to fabricate the housing equipped with the microneedles. Analytical characterization of the flow rate through the microneedles showed an outgoing flow rate ranging from 63 μL/min to 520 μL/min for an applied pressure of 0.1 to 1 kPa. In one embodiment, the assembled system has an overall size of 3.9 mm×2.1 mm×2 mm and this system achieved a delivery of 4±0.5 μL within 12 seconds of actuation. A penetration test of the microneedle into a skin-like material confirms its potential for transdermal delivery.

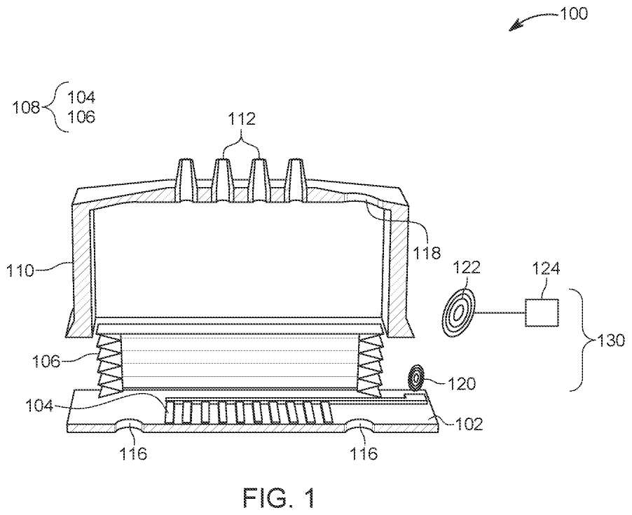

A miniaturized delivery system 100 is illustrated in FIG. 1 and includes a base or substrate 102 (for example, a printed circuit board or a flexible substrate that conforms to the human body) on which one or more interdigitated electrodes 104 are formed. A bellows membrane 106 is attached to the substrate 102, and is configured to enclose the electrodes 104. In one embodiment, the bellows membrane 106 is directly attached to the substrate 102. The electrodes 104 and bellows membrane 106 form the electrolytic pump 108. A housing 110 is placed over the bellows membrane 106 and is attached to the substrate 102 so that the bellows membrane 106 is insulated from an ambient. In one embodiment, the housing 110 is directly attached to the substrate 102. In one application, the bellows membrane 106 is not in contact with the housing 110 when the bellows membrane is in a retracted state. However, the bellows membrane may touch the housing when in an extended state. The housing 110 is formed with one or more microneedles 112. One or more pump purge ports 116 are formed in the substrate 102 and a drug refill port 118 is formed in the housing 110 for reasons discussed later.

Bellows membranes are suitable as actuators for micro-electrolytic pumps due to their fitting pressure ranges. The predictable performance and expansion profile of micro-bellows translates into the controlled dosage in drug delivery. Due to micro-bellows' malleability, they can expand under internal pressure, making them ideal membranes for an electrolytic pump. Combined with Parylene C's biocompatibility, they act as a diaphragm isolating the drug reservoir housing 110 from the pumping source, thus preventing degradation and pH changes from water exposure. Due to these pumps' minimal power requirements, wireless inductive powering units can be installed to achieve wireless actuation. With an electromagnetic field from a transmitting coil, a current may be induced in the receiving coil for driving the electrolytic reaction in the pump.

Traditional fabrication and design methods used to limit the pump to be operated in very specific scenarios. With additive manufacturing methods such as two-photon polymerization, rigid applications can be avoided. Following the same process, with minimal dimensional design edits, a set of versatile pumps can be fabricated and implanted at different sites, paving the way towards novel therapeutic options. Combined with the use of microneedles, these integrated systems have a promising potential for targeted drug delivery for the treatment of tumors and critical diseases like atherosclerosis.

In this embodiment, the system 100 is a miniaturized and wirelessly powered drug delivery system. The system 100 may further include a receiver coil 120 that is attached to the electrodes 104. To power the receiver coil 120, an external inductive powering unit 130 may be used. The inductive powering unit 130 may include a transmitter coil 122, which is connected to a power source 124. The power source 124 may be a battery, a fuel cell, a solar panel, an electronic power source that is connected to the power grid, a computer, a mobile device, etc. The electrical power generated by the power source 124 is transferred to the transmitting coil 122. With this electrical energy, the transmitting coil 122 generates a magnetic flux, which is received by the receiver coil 120. The receiver coil 120 transforms the magnetic flux back into electrical energy, which is then distributed to the electrodes 104.

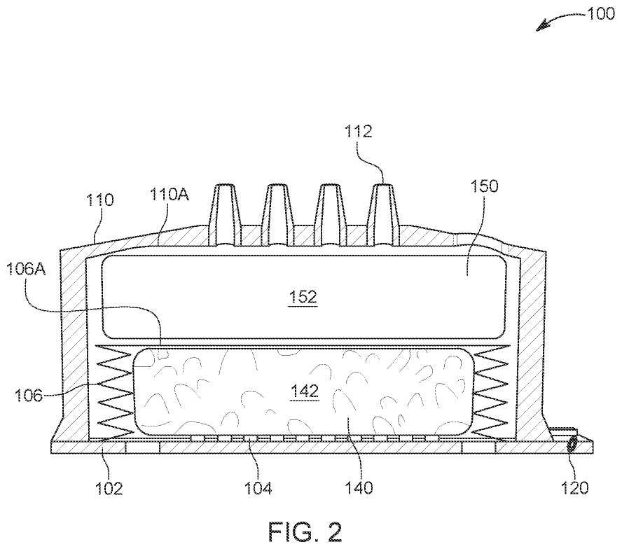

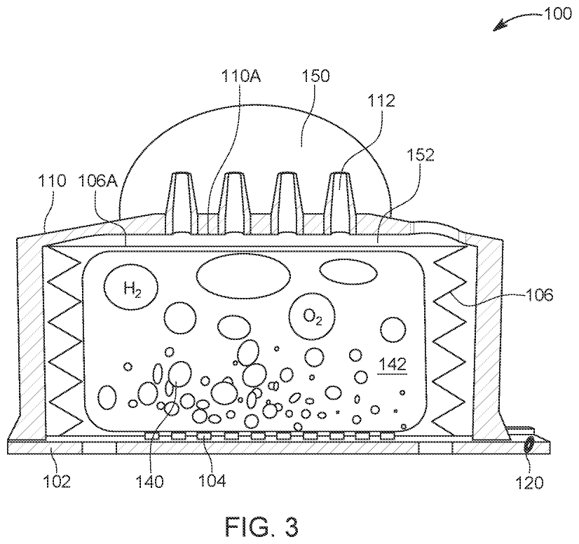

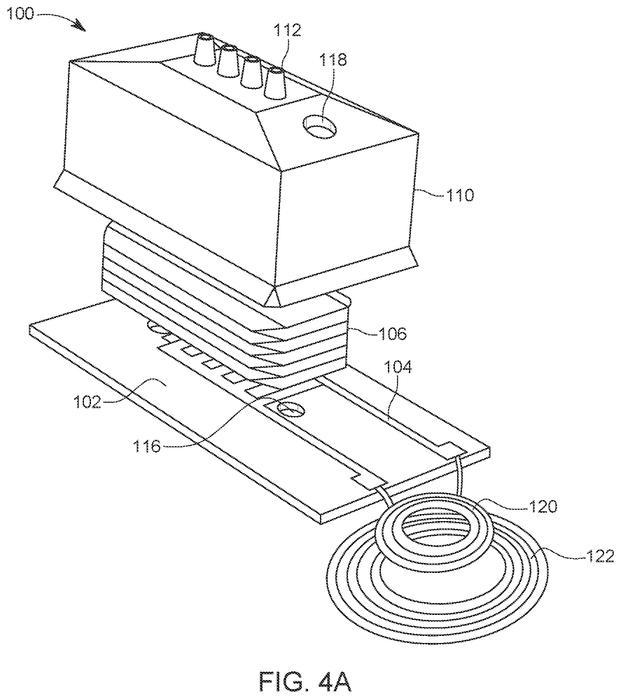

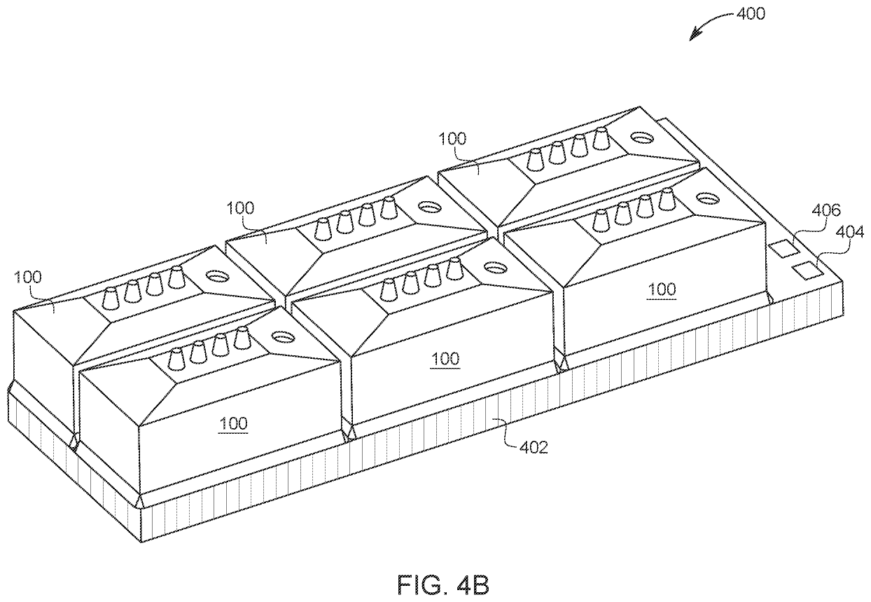

The electrodes 104 are in direct contact with a first fluid 140 (e.g., water) that is stored in a first internal chamber 142, as shown in FIG. 2. The first internal chamber 142 is defined by an interior face of the bellows membrane 106 and the substrate 102. Note that the digitated electrodes 104 are situated inside the first internal chamber 142, as shown in FIG. 2. The energy received from the receiver coil 120, is used for electrolysis by the electrodes 104. As a result of the electrolysis process, hydrogen and oxygen gasses are produced at the interface of the first fluid 140 and the electrodes 104, when the first fluid is water. As a result of these two gases, a volume of the first fluid 140 inside the first chamber 142 is increasing, which makes the micro-bellows membrane 106 to expand from the retracted state of FIG. 2, to an extended state as illustrated in FIG. 3, and to push out a second fluid 150, which is stored in a second internal chamber 152. The second internal chamber 152 is defined by an internal face 110A of the housing 110 and an external face 106A of the bellows membrane 106, as illustrated in FIG. 2. Whiles FIGS. 1-3 shows a cross-section through the delivery system 100, FIG. 4A shows a perspective view of the system.

In one embodiment, as illustrated in FIG. 4B, an array 400 of delivery systems 100 is distributed on a same substrate 402, and controlled by a processor 404. Each delivery system can be loaded with a different substance to be delivered, and each delivery system can be controlled independent of the others by the processor 404, which is located on the substrate 402. Thus, multiple doses (of the same or different materials) can be loaded on the array 400 and distributed synchronously or not to the patient. In one application, the processor 404 is a global processor that communicates with each of the delivery system 100. In one application, a power supply source 406 may be present on the substrate 402 for supplying the necessary energy to activate the delivery systems 100 and/or the processor 404. In one application, the power supply source 406 is a battery or a coil or other similar device.

Note that the bellows membrane 106 is designed to expand until the top surface 106A of the membrane directly contacts the housing 110, as shown in FIG. 3. In one embodiment, the external face 106A of the bellows membrane 106 remains flat while pressing against the internal face 110A of the housing 110, which is also flat. Thus, when the external flat face 106A of the bellows membrane 106 touches the internal flat face 110A of the housing 110, as illustrated in FIG. 3, a volume of the second internal chamber 152 is substantially reduced to zero, so that the entire fluid 150 from the second internal chamber 152 can be expelled of the delivery device 100. While this embodiment refers to a flat face 110A and a flat face 106A, in another embodiment, one or both of these faces may be curved.

In one embodiment, the external face 106A of the bellows membrane 106 is substantially parallel to the internal face 110A of the housing 110. In one application, the bellows membrane 106 has a retracted state, as shown in FIG. 2, in which the external face 106A is farthest from the internal face 110A of the housing 110, and an extended state, as shown in FIG. 3, in which the external face 106A is closest to the internal face 110A of the housing 110. In one application, the external face 106A of the bellows membrane 106 is substantially parallel to the internal face 110A of the housing 110 for both the retracted state and the extended state.

In another embodiment, the delivery system 100 shown in FIGS. 1 to 4 does not have an active intake port that provides the drug to the second internal chamber while the system is attached to the skin or organ. In other words, the delivery system is supplied with the drug through the intake port 118 once, after which that port is sealed and the system is attached to the person. While attached to the person, no drug is supplied to the system through the intake port 118 or any other port. The only way for the drug in or out of the housing 110 is through the needles 112, and the drug is dispensed out of the housing 110, through the needles 112, directly into the organ (the organ of a person is understood herein to include the skin, any actual organ, a blood vessel, etc.) of the person. For this reason, the bellows membrane 106 moves only from the retracted state to the fully extended state, and does not move back and forth between these two states.

Each of the components of the system 100 are now discussed in more detail. In one embodiment, the housing 110 and the microneedles 112 are integrally manufactured by 3D printing using a two-photon polymerization (TPP) technique. The microneedles (MNs herein) used for transdermal delivery have to overcome the skin's mechanical resistance by piercing the stratum corneum and penetrate up to the dermis layer without mechanical failure. In this regard, the prediction of the forces applied to the MNs needs to be known. Because the MNs feature high aspect ratios and low tapering angles, they are mainly prone to buckling and fracture. The yield failure known as the fracture is due to an applied load higher than the yield strength of the MN material, whereas the buckling failure leads to deformation of the MNs into an arched shape. To predict the buckling force applied on the MNs, an analytical model derived by Kim et al. (K. Kim, D. S. Park, H. M. Lu, W. Che, K. Kim, J.-B. Lee, C. H. Ahn, J. Micromechan. Microeng. 2004, 14, 597) for the fixed-free tapered hollow truncated cone structure was used. The estimation of the fracture force was based on the assumptions that the failure or fracture of the MN is caused by axial forces applied to the MN tip, which means that shear forces are neglected and that the MN fracture is mainly due to an applied pressure higher than the ultimate stress of the material. The MN penetration force into the human skin has been investigated by Davis et al. (S. P. Davis, B. J. Landis, Z. H. Adams, M. G. Allen, M. R. Prausnitz, J. Biomech. 2004, 37, 1155) and Khanna et al. (P. Khanna, K. Luongo, J. A. Strom, S. Bhansali, J. Micromech. Microeng. 2010, 20, 045011) and thus, the MN insertion force into the human skin and the skin toughness are data. To predict the required force for a MN to pierce the human skin, Davis et al. have developed an empirical expression of the insertion force based on the puncture fracture toughness Gp and the MN geometry, and this equation has been used by the inventors to fabricate (configure) the housing 110 and needles 112 to prevent failure when inserted into an organ.

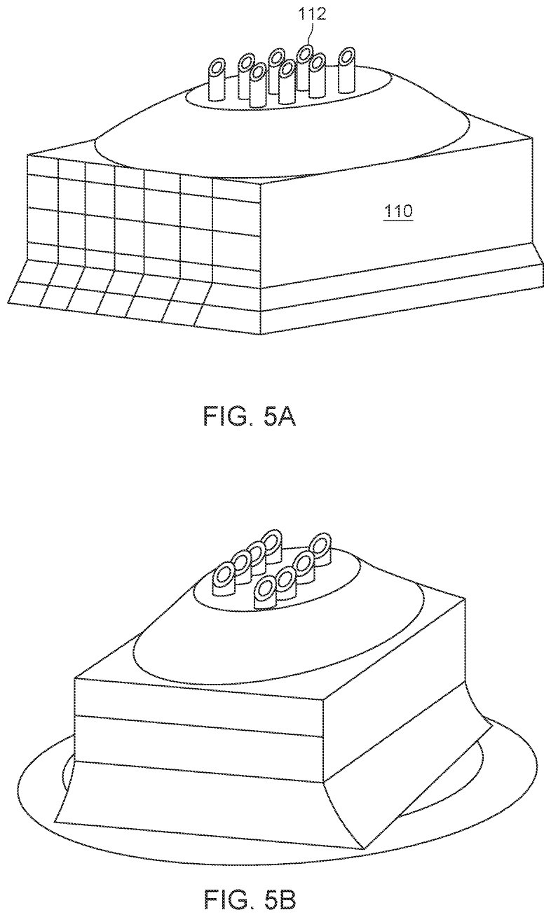

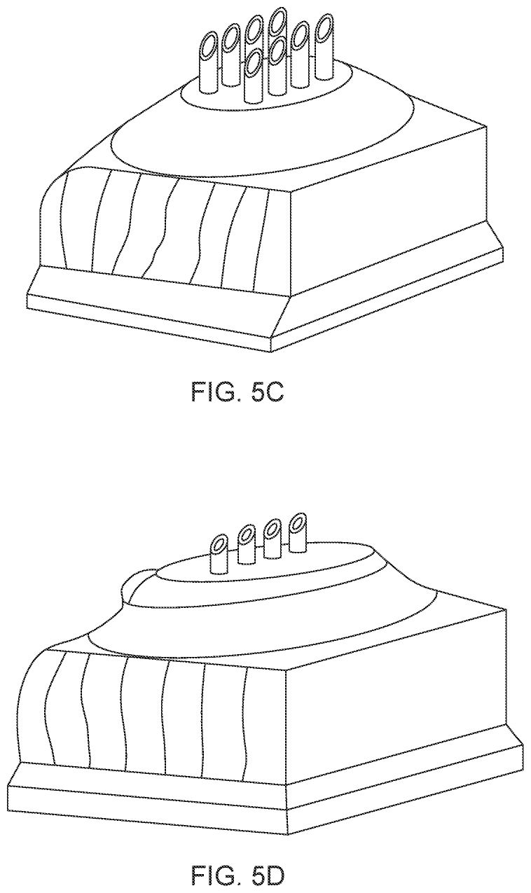

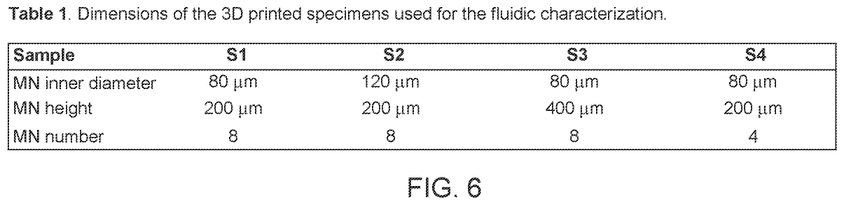

The microchip design consisted of a drug reservoir 110 with an array of MNs 112 formed on top of it, as illustrated in FIGS. 5A to 5D. The reservoir dimensions were 2×1×1 mm³. Four different designs were fabricated, as illustrated in Table 1 in FIG. 6. Sample S1 (shown in FIG. 5A) had an array of eight MNs arranged in two rows of four MNs each, where the MNs' diameter and height were 80 and 400 μm, respectively. The other designs shown in Table 1 had either a larger MN diameter (S2, FIG. 5B), a longer MN height (S3, FIG. 5C), or a lesser number of MNs (S4, FIG. 5D) compared to (S1). The slicing distance, which is the distance between the layers in the vertical direction, was set to 2 μm. The hatching distance, which is the distance between two lines of the laser beam in the horizontal plane, was set to 2 μm. Both parameters (slicing/hatching) were chosen after an optimization process to fabricate a robust structure in a short period of time.

The 3D printing fabrication process started with a 500 μm-thick single-side polished silicon substrate. Prior to the 3D printing step, an elliptical/rectangular void (minor axis 0.8 mm and major axis 1.5 mm) was etched through the silicon substrate using a 50 W fiber laser (PLS6MW, Universal Laser Systems GmbH, Vienna, Austria) with 1.06 μm wavelength. Then, the substrate was cleaned in an ultrasonication bath of acetone and isopropyl alcohol. Subsequently, it was washed with deionized (DI) water and dried using a gentle stream of nitrogen gas. IP-S photoresist (Nanoscribe GmbH, Germany) was then drop cast on the center of the silicon substrate, on top of the elliptical hole, and loaded into the Nanoscribe Photonic Professional GT laser lithography system (Nanoscribe GmbH, Germany). The designed structure was printed layer by layer in a dip-in laser lithography configuration. The objective lens (25× magnifications and NA ¼ 0.8) was immersed in the resist and focused on the silicon interface, then, positioned at the void center. IP-S was chosen for its low shrinkage effect, smooth surfaces, and ability to print feature size ranging from the submicron to the millimeter scale.

Polymerization of the photoresist was induced by the laser at 780 nm wavelength, 100 mW power, and 50 mm s⁻¹ scan speed. Following the printing process, the 3D printed assembly (reservoir and MNs) was developed by immersion in mr-DEV 600 (microresist technology GmbH, Germany) for 10 min to remove the unpolymerized excess of resist. Then, to clear the MNs channels, the sample was immersed again in the developing solution under vacuum for 15 min. Subsequently, it was immersed in isopropanol (IPA) for an additional 5 min to remove the residual photoresist and dilute the remaining developing solution. Finally, the sample was dried with a gentle stream of nitrogen gas. This process was applied to fabricate the four different samples, i.e., S1, S2, S3, and S4 illustrated in FIGS. 5A to 5D.

To test the MNs on a material that has skin-like mechanical properties, PDMS samples were created with an elasticity modulus equal to or higher than the one of the human skin. The mechanical properties of the human skin were investigated in vivo by Liang and Boppart (X. Liang, S. A. Boppart, IEEE Trans. Biomed. Eng. 2010, 57, 953) for different locations of the human body and for different dehydration levels of the skin. They found that the elasticity modulus varies from 0.1 to 0.3 MPa. In the case of PDMS, the elasticity modulus is linearly dependent on the crosslinking ratio (from 5:1 to 33:1), with values between 3.6 and 56 MPa, respectively. A crosslinking ratio of 10:1 (base/curing agent) was used to prepare PDMS skins (Sylgard 184 Silicone Elastomer, Dow Corning Corp., Midland, Mich., USA), corresponding to an elasticity modulus of 2.6 MPa, which is about an order of magnitude higher than the Young's modulus of human skin. Using an Electromechanical Testing System, a single MN was attached to the indenter, and a PDMS skin was placed on top of a support. The PDMS skins were prepared by drop casting with 700 and 160 μm of thickness, depending on the height of the MN (1000 and 200 μm long, respectively). The insertion rate was 5 μms⁻¹.

To evaluate the penetration depth of the MNs array and validate delivery of a liquid solution into the skin, a fluorescent dye was injected through the MNs into a mouse's skin. Before testing, a hollow (1 mm in diameter) acrylic sheet (10 by 10 and 1 mm thickness) was cut using a laser cutter (Universal PLS6.75 10.6 μmCO2). Then, a 19G blunt tip needle and the MNs array were glued to the back and front sides, respectively, of the acrylic substrate using super glue. With this assembly, the MN array was applied manually on the back and chest of a euthanized female nude mouse (10 months old, CD-1 nude mouse, Charles River laboratories). Using a 1 mL syringe connected to the 19G needle, fluorescein isothiocyanate (FITC) (Sigma Aldrich, USA) dye was injected. The mouse skin was then excised and imaged using a Leica SP8 inverted confocal microscope (Leica, Germany) with a 10× objective. The MN samples used in the mouse skin penetration experiment were S1 and S3 (200 and 400 μm long, respectively) (Table 1). For each MN type, five samples were tested. Flow rate measurements and a cytotoxicity test were performed for these samples.

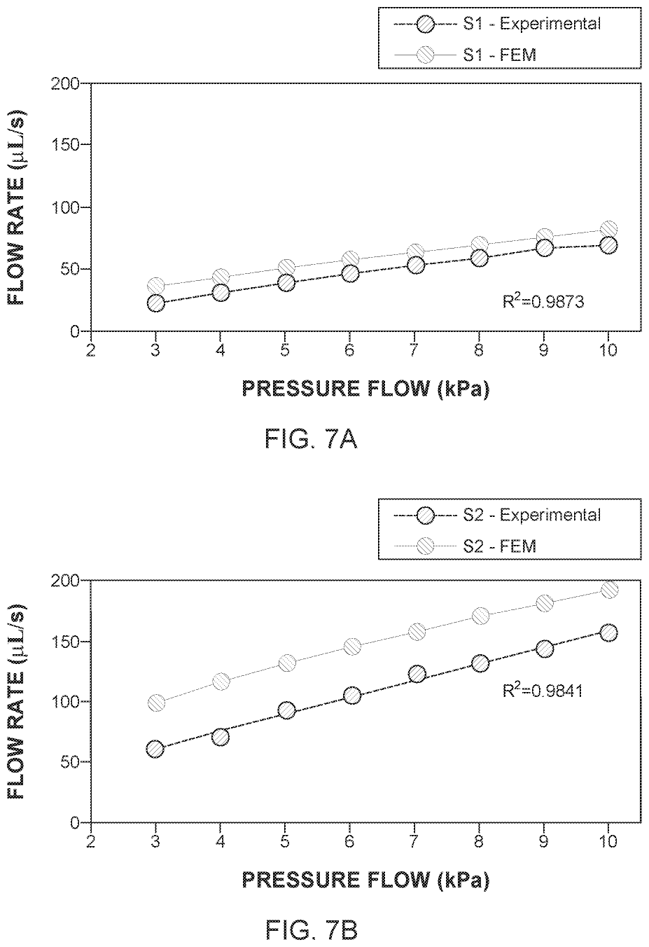

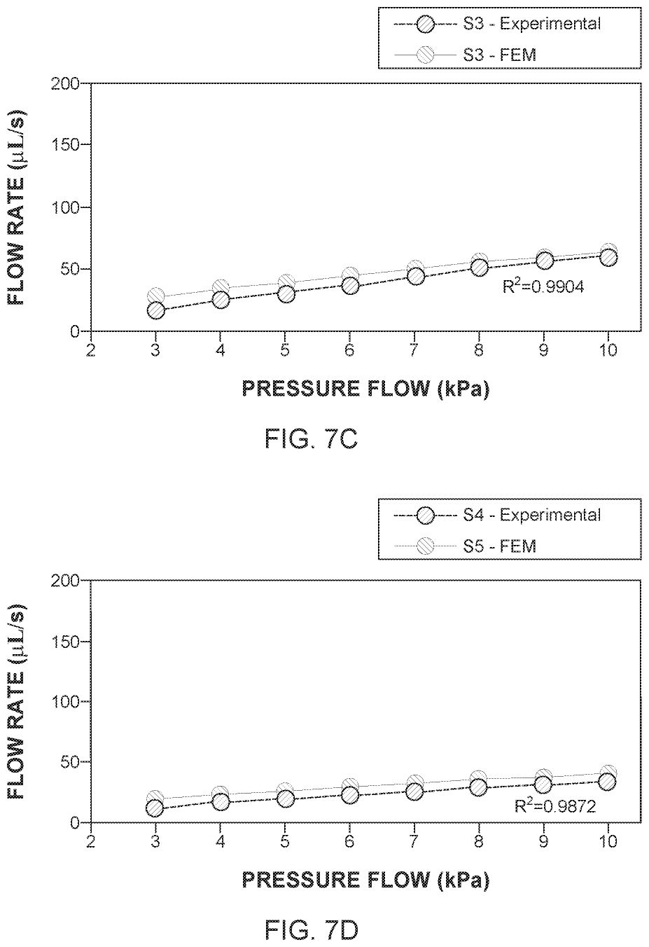

The flow rate as a function of pressure is shown for all samples in FIGS. 7A to 7D. All experimental results showed an excellent linear fit (R2 ranging between 0.99 and 0.98) and agreed well with the FEM simulation. This linearity of the experimental result suggests a fully developed laminar flow through the needle bores, which is also corroborated by the calculated Reynolds numbers, which ranged between 30 and 230. The experimental flow rates through sample S1 (FIG. 7A), which has 8 MNs with a diameter of 80 μm and a shaft length of 200 μm, are approximately double that of sample S4 (FIG. 7D), which has four needles of the same dimensions, through the entire pressure range. As suggested in the art, the experimentally acquired flow rates support the hypothesis that the aggregate flow through the array is a result of independent flow rates through N identical MNs that experience the same drop in pressure. The spacing of the needles in the array minimizes cross influence between the independent flows. As a result, the flow profile can be modulated by maintaining the spacing and adjusting the needle count. The impact of bore radius and shaft length on the flow rate is notable when comparing sample S1 (FIG. 7A) to sample S2 (FIG. 7B) and sample S3 (FIG. 7C), respectively. Increasing the radius from 80 μm (S1) to 120 μm (S2) nearly doubled the flow rate. Similarly, increasing the shaft length from 200 μm (S1) to 400 μm (S3) reduced the flow rate by 33%.

The tensile test on the 3D printed IP-S bars allowed the determination of the stress-strain curve, from which the elasticity modulus and the yield strength were extracted. The sample with 1 μm of slicing/hatching distances has stronger mechanical properties. The elasticity modulus and yield strength are 1740+/−15 and 100+/−2.8 MPa, respectively, for the samples with 1 μm of slicing/hatching distances, and they are equal to 867.27+/−27.04 and 64.58+/−5.74 MPa, respectively, for the sample with 2 μm of slicing/hatching distances. Decreasing the slicing and hatching distances resulted in denser structures, which had about two times stronger mechanical properties. This suggests that the material strength and elasticity can be tailored to intermediate properties by modifying the printing parameters, particularly the slicing and hatching distances.

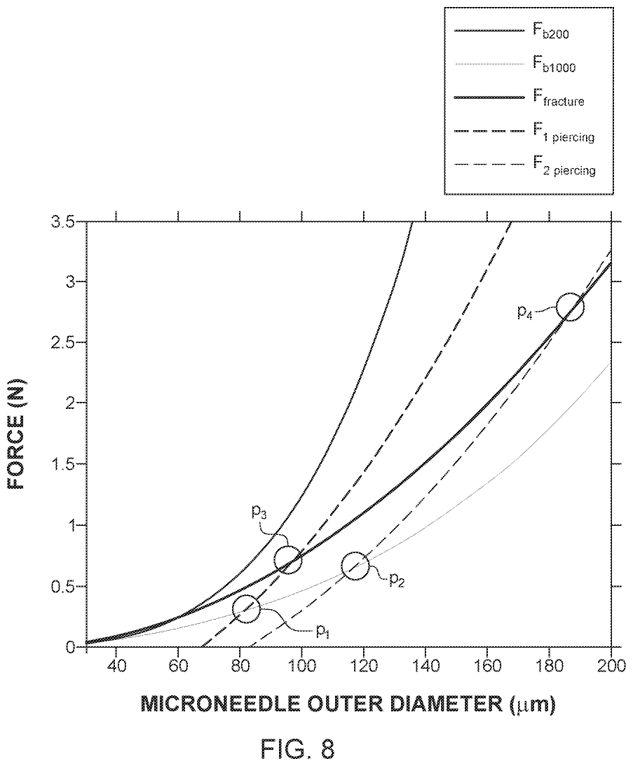

The buckling forces were estimated for two different MN heights, 200 and 1000 μm, represented by Fb200 and Fb1000, respectively, as shown in FIG. 8. The skin puncturing force was assessed for the two reported fracture toughness limits Gp1 and Gp2, which correspond to a hard and soft skin and are shown in FIG. 8 by the dashed lines for F1 piercing and F2 piercing, respectively. Generally, with increasing the MN's outer diameter, the buckling force increases. The buckling effect is stronger for the 1,000 μm-long MN regardless of the MN diameter. The 1,000 μm-long MN will always buckle before a fracture occurs, whereas in case of the 200 μm-long MN, the fracture force is higher than the buckling force only for an outer diameter less than 60 μm. For bigger diameters, the MN will face fracture before buckling. The minimum required force to puncture the skin for a MN with an outer tip diameter less than 80 μm is estimated to be less than 0.01 N based on the theoretical analysis shown in FIG. 8. In case of the 1,000 μm-long MN, the hard skin can be penetrated without buckling, when the MN diameter is smaller than 80 μm (see critical point p1 in FIG. 8), whereas the soft skin can be penetrated when the diameter is smaller than 115 μm (see critical point p2 in FIG. 8).

Nevertheless, the penetration into the skin is still possible by applying additional force, while not reaching the fracture force limits (see point p3 for the hard skin and p4 for the soft skin in FIG. 8). Therefore, for a 1,000 μm-long IP-S 3D printed MN, the maximum outer diameter has to be less than 80 μm to puncture the human skin without any mechanical failure (considering the hard skin). Moreover, regardless of the MN height, the MN tip diameter has to be less than either 95 or 180 μm based on the type of skin; otherwise, the MN will break before penetrating the skin. The skin penetration of the 200 μm-long MN is limited by fracture only, with the same values as the 1,000 μm-long MN.

A penetration test indicated that both MNs (200 and 1,000 μm) were able to puncture and penetrate the PDMS layers without mechanical failure. The 200 μm-long MN penetrates the 160 μm-thick PDMS layer at an applied force of 0.095 N and after displacement of about 118 μm. Before puncturing, the PDMS layer was deformed and buckled, due to its elasticity. Similarly, the 1,000 μm-long MN penetrates the 700 μm-thick PDMS layer at an applied force of 0.115 N and after displacement of about 480 μm. After puncturing, the force remains constant until it increases again, due to the direct contact of the MNs with the support under the PDMS layer. Although the tip geometry is similar for the two MNs, the piercing forces were slightly different (0.095 and 0.115 N for the 200 and 1,000 μm-long MNs, respectively) due to the difference in the PDMS layer thickness (160 and 700 μm for the 200 and 1,000 μm-long MNs, respectively).

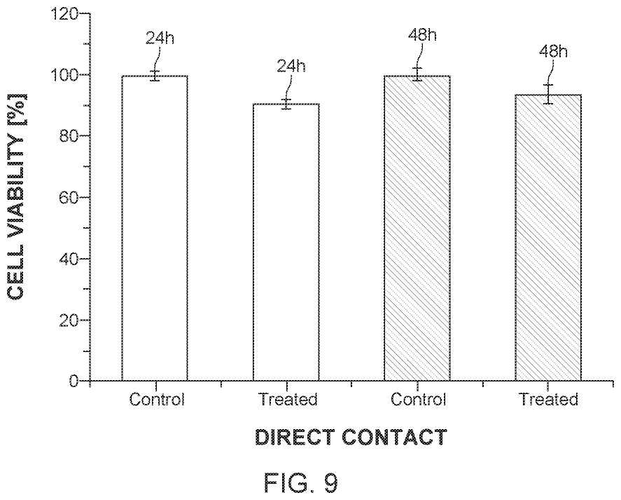

The results of the cytotoxicity test illustrated in FIG. 9 show a decrease in cell viability of no more than 10% for cells grown on top of cured IP-S resin for 24 and 48 h. Furthermore, the LIVE/DEAD™ viability assay confirms the growth of the cells on the resist substrate. According to ISO 10993-5 (part 8.5 determination of cytotoxicity), a cytotoxic effect is present, in case of a 30% reduction in cell viability. Hence, the IP-S resin can be considered not toxic in these experiments, even after 48 h of direct contact exposure. In the experiments where the growth medium was exposed to the cured resin, and later used for cell culture, the decrease in cell viability is again low with less than 30% in all cases. There is, however, a more significant decrease in viability compared with the experiments, where the cells were grown directly on IP-S resin. The largest reduction was found for the experiments, where the extraction vehicle was kept in a thermal mixer. This might be due to an extra step in the extraction process in the thermal mixer, as the mixer was kept running at 500 rpm, which can provide homogeneity. Another reason can be the change of pH in the extraction vehicle that was not kept in appropriate CO2 conditions. A McCoy 5A modified medium requires 5-10% CO2 levels to maintain physiological pH conditions. If not supplied, the sodium bicarbonate buffer system that this medium possesses may lose its buffering effect and shift the medium's pH toward alkalinity values, which are not suitable for healthy cell growth. In any case, the results indicate that the cured IP-S resin has a little negative effect on cell proliferation as the viability is steadily kept at more than 70% and is biocompatible.

The inventors determined that the use of the high-resolution TPP 3D printing technique allowed for the robust and seamless integration of MNs with a chamber or delivery systems, for biomedical applications, circumventing the need for laborious and complex fabrication techniques. A reservoir 110 of 2 mm³ volume topped with hollow MNs 112 with inner diameter and height ranging from 30 to 120 μm and from 200 to 1000 μm, respectively, can be fabricated as discussed above. Note that the dimensions of the reservoir 110 are not limited to the numbers noted above, but they may be customized depending on the delivery application, the amount needed to be delivered, the type of disease or condition to be addressed, so that a personalized treatment for a given subject can be achieved. The outgoing flow rate through MNs using FEM and experiment for four different designs has determined that the flow profiles are laminar at an applied pressure range of 3-10 kPa. By modifying the MNs count, diameter, and shaft length, the flow rate can be modulated from 20 to 160 μLs⁻¹. An additional analysis of the mechanical properties of the IP-S photoresist used to print the MNs has determined the elastic modulus and the yield strength of the solid resist, which were 852-1750 and 65-102 MPa, respectively. Using these mechanical properties, the buckling and fracture forces of the MNs were derived. Combined with experimental testing, this analysis verified the appropriate dimensions of the MNs that are needed to ensure mechanical stability for a given application. To corroborate the applicability of the 3D printed MNs, they were used for a penetration test into both a skin-like material and mouse skin. Penetration into skin-like material allowed the determination of the piercing force which was 0.095-0.115 N. Confocal microscopy of the mouse skin confirmed the MN array penetration and fluorescent dye delivery 100 and 180 μm deep into the skin for the 200 and 400 μm-long MNs, respectively. A complementary biocompatibility assessment was performed to investigate the potential of using the technique for direct tissue interfacing or implants, and it has determined that the photoresist has minimal cytotoxicity, which makes it ideal for such applications.

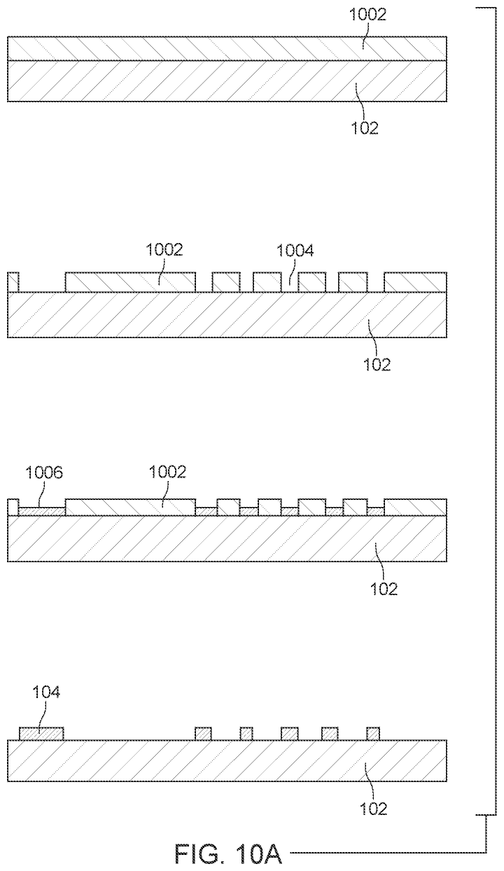

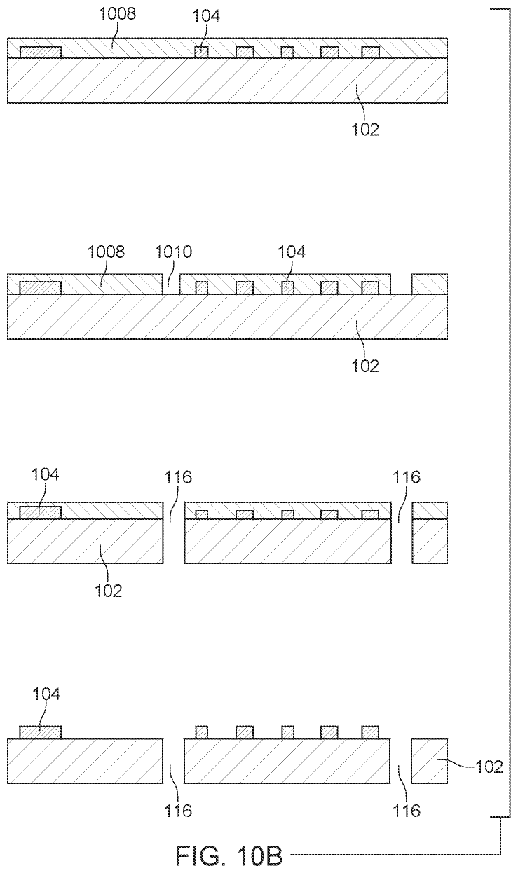

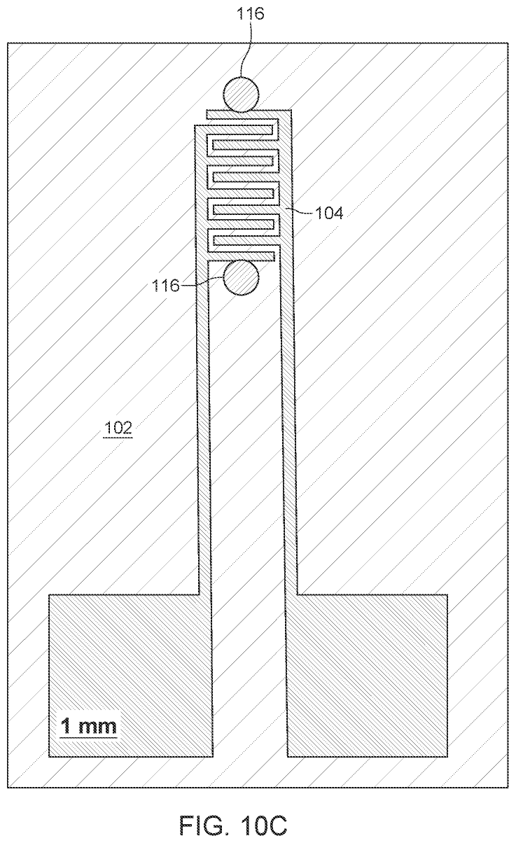

The electrolytic pump 108 of the system 100, as previously discussed, includes the electrodes 104 and the bellows membrane 106. In one embodiment, the pump 108 may also include the substrate 102. The interdigitated electrodes 104 were made in one embodiment as 5 finger pairs (100 μm/100 μm elements width/spacing) with a total area of 1.25 mm². They were fabricated on a silicon substrate 102, as now discussed with regard to FIGS. 10A to 10C. A liftoff process using AZ ECI 3027 (MicroChemicals GmbH, Ulm, Germany) photoresist was employed to pattern the Ti/Pt (30 nm/300 nm) electrodes as shown in FIG. 10A. The process involved spinning a photoresist 1002 on the Si substrate 102, patterning the photoresist 1002 to form holes 1004 on top of the substrate 102, depositing a metal 1006 inside the holes 1004, and removing the left photoresist to obtain the electrodes 104 formed on top of the substrate 102.

Two holes 116 (300 μm in diameter) were created through the silicon substrate 102 by Deep Reactive Ion Etching following the process described in FIG. 10B using the photoresist AZ 9260 (MicroChemicals GmbH, Ulm, Germany). More specifically, the photoresist 1008 was deposited on top of the electrodes 104, then the photoresist 1008 was patterned to form channels 1010, directly above parts of the Si substrate 102. These parts of the substrate 102 were etched to form the holes 116, and then the photoresist 1010 was removed to expose the electrodes 104. The holes 116 served to inject the electrolyte solution (e.g., 1 wt % NaCl solution in DI water) between the interdigitated electrodes 104 and micro-bellows membrane 106, into the first chamber 142. FIG. 10C shows a top image of this electrodes 104 and ports 116 formed on the substrate 102.

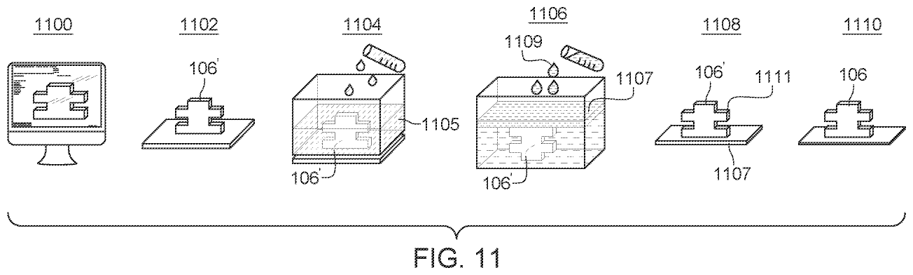

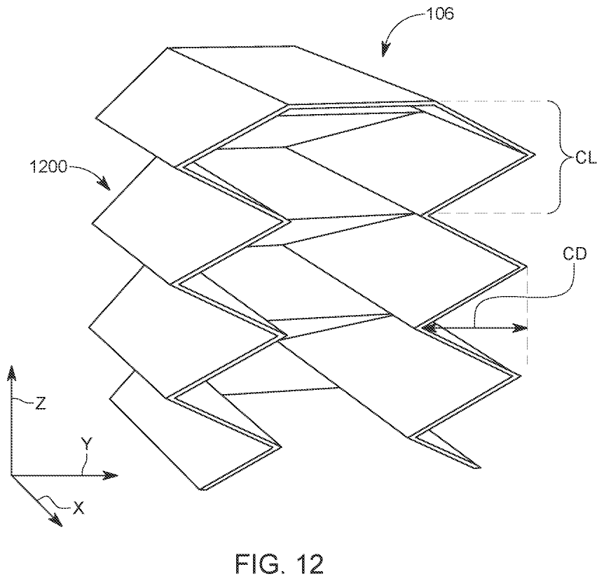

The fabrication process of the micro-bellows membrane 106 (based on Parylene C) is summarized in FIG. 11, and includes a step 1100 of designing the membrane, a step 1102 of 3D printing a negative replica 106′ of the membrane 106, which serves as a master mold, a step 1104 of PDMS 1105 casting the membrane, a step 1106 of adding a sacrificial mold 1107 to the PDMS 1105 by using melted wax 1109, a step 1108 of removing the replica 106′ and coating it with Parylene C 1111, and a step 1110 of releasing the membrane 106 from the replica 106′. This process is discussed in more detail in [4]. The membrane 106's dimensions in this embodiment are 3 mm by 1.2 mm in length and width, respectively, with a height varying from 0.5 mm, when the membrane is fully folded (deflated), to about 2 mm, when it is fully expended (inflated). This large deflection is facilitated by outlying triangular corrugations 1200 (250 μm of corrugation depth and length) illustrated in FIG. 12. Note that CL in FIG. 12 indicates the corrugation length and CD is the corrugation depth. The number of corrugations is between 1 and 10, the corrugation length is about 100 to 500 μm (for example, 300 μm) and the corrugation depth is about 50 to 400 μm (for example, 250 μm).

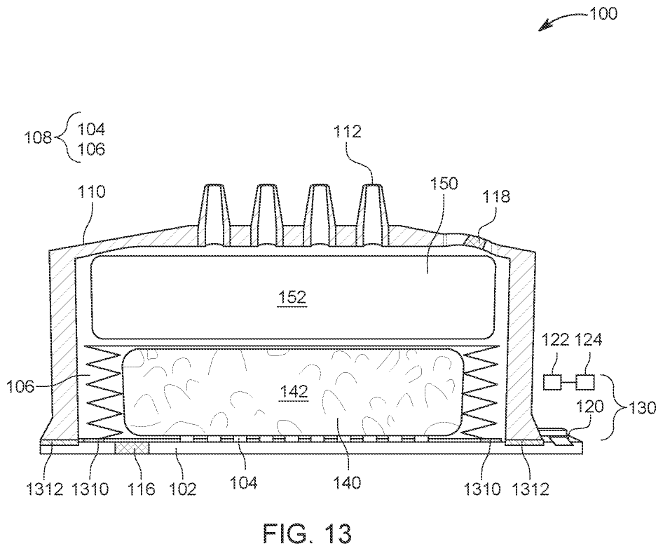

Assembly of the delivery system 100 is now discussed with regard to FIG. 13. The pumping mechanism 108 is based on the inflation of the Parylene C micro-bellows membrane 106, due to gas bubbles generated from the water electrolysis reaction in the first chamber 142. Such an actuation mechanism has a significant volume change even in a pressurized medium and low power consumption. The micro-bellows membrane 106 was assembled on top of the interdigitated electrodes 104 by being glued to the substrate 102, with a glue 1310, as illustrated in FIG. 13. The membrane 106 is so attached to the substrate 102 that no fluid 140 escapes from the first chamber 142. In one embodiment, the membrane is shaped as a bag having a single side open, and this side is fully attached to the substrate.

Then, about 1.5 μL of 1 wt % NaCl solution in DI water was injected inside the first chamber 142 of the membrane 106, through the port 116. The port 116 was then sealed, for example, with tape. Then, the 3D printed housing 110 was assembled on top of the electrochemical pump 108, by gluing the housing 110 directly to the substrate 102, for example, with a glue 1312, that may be the same as glue 1310 or different. The housing 110 completely seals the pumping mechanism 108 and also forms the second chamber 152 so that no fluid 152 escapes from the second chamber. The first and second chambers do not fluidly communicate with each other. The second chamber 152 is then filled with the liquid drug 150 through the refill port 118, which is then sealed. Thus, at this stage, the delivery system 100 has no input or output port, except for the needles 112.

The power transmission unit 130 was implemented in the embodiment illustrated in FIG. 13 as a wireless transmission module having a transmitting coil 122 (33 mm outer diameter, 5 mm inner diameter and 1 mm thickness). The module was powered with a DC voltage generator 124 (e.g., 5V, 0.1A). The transmitter coil provided an output voltage and current of ˜10 mA and 5V, respectively, in the receiver's coil 120 (21 mm outer diameter, 10 mm inner diameter and 0.5 mm thickness) at a 10 mm distance. When connecting the receiver coil 120 to the interdigitated electrodes 104, water electrolysis initiates and oxygen/hydrogen bubbles are produced, as illustrated in FIG. 3. Consequently, the bellows membrane 106 is inflated pushing the drug 150 through the microneedles 112. In one embodiment, the membrane 106 reached 1.9 mm of total deflection starting from an initial height of 0.5 mm, thus achieving about 300% expansion. The micro-bellows membrane 106's geometry and expansion determined the maximum volume to be delivered. In this case, for an expansion of 1.2±0.1 mm, the amount delivered was 3.8±0.3 μL. For this configuration, it is possible to deliver ˜3.8 μL of fluid 150 through the microneedles 112 within 10 seconds of actuation.

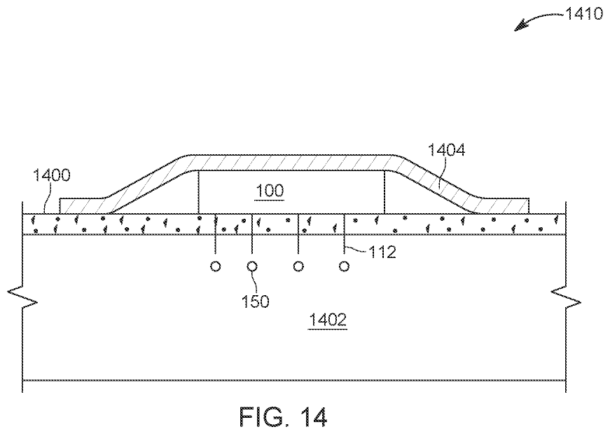

The miniaturized delivery system 100 can be attached to the skin 1400 of a human 1402 so that one or more of the microneedles 112 directly penetrate the skin and thus, as shown in FIG. 14, the drug 150 can be pumped directly under the skin. To secure the delivery system 100 to the skin 1400, a band-aid 1404 or similar means (e.g., a piece of tape or a drop of glue) may be placed over the delivery system and over the skin. Note that it is possible to attach the system 100 directly to the skin 1400 with just a band-air due to the very small size of the delivery system 100, for example, 3.9 mm×2.1 mm×2.0 mm. For this configuration, the delivery system 100 can be attached to any part of the human body, or even to an animal skin. Of course, if the delivery system 100 is used for non-medical purposes, it can be attached to other objects than a human or an animal, for example, to a plant, a bush, a tree, etc. The delivery system 100 and the means 1404 for attaching the delivery system to the skin or organ form a delivery system kit 1410.

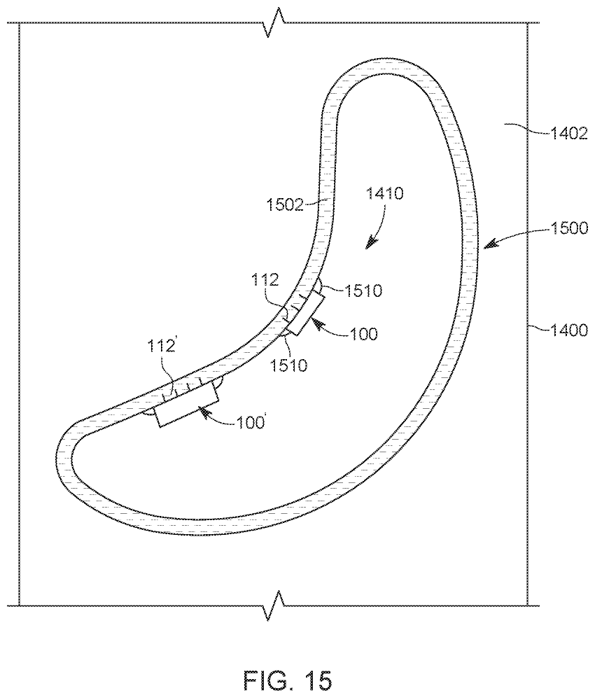

In another embodiment, the delivery system can be attached internally to the human body, i.e., directly to an organ or a vessel as illustrated in FIG. 15. More specifically, FIG. 15 shows a part of a human body 1400 (the torso) and a stomach 1500. The stomach 1500 has a wall 1502 and the delivery system 100 is attached with ligatures 1510 (which may be part of the means 1410) directly to the wall 1502. The needles 112 of the delivery system 100 are directly attached to the wall 1502. While FIG. 15 shows the delivery system directly attached to an inside of the wall 1502 of the stomach, the system may be attached to an outside of the wall, to any other organ, to a blood vessel, or even to an inside of the skin, as long as the receiver coil 120 is in range of the transmitter coil 122 so that electrical energy can be transmitted to the electrodes 104. In one embodiment, the receiver coil 120 can be replaced with a small battery and a processor. For this configuration, the processor can be programmed to connect the electrodes 104 to the battery at certain times, so that only at those times the pumping mechanism 108 is actuated, and the drug fluid 150 is released. Once the battery is spent, the entire delivery system 100 can be removed from the body and disposed. The same is true with the delivery system that is provided with the receiver coil 120. In one embodiment it is possible to have additional delivery systems 100′ attached to the organ, similar to the delivery system 100, to deliver an additional amount of the drug or a different drug. In one embodiment, the additional delivery systems 100′ may be formed on the same base as the delivery system 100.

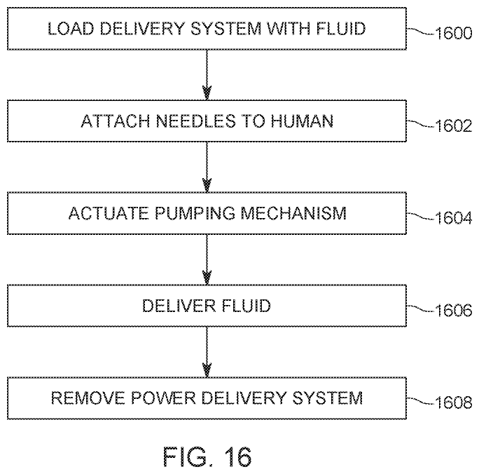

A method for delivering a drug to a person with the delivery system disclosed above is now discussed with regard to FIG. 16. In step 1600, the second chamber 152 of the delivery system 100 is loaded with the desired drug 152. In step 1602, the microneedles 112 of the delivery system 100 are directly attached to the skin or any other organ of a person. In step 1604, a power delivery system is brought next to a receiver coil of the delivery system 100 for actuating a pumping mechanism 108 of the delivery system. In step 1606, the delivery system 100 delivers a certain amount of the drug 150 directly into the skin or other organ of the person. In step 1608, the power delivery system is removed so that the delivery of the drug 150 is stopped.

The disclosed embodiments provide a miniature delivery system that has needles that directly attach to the human body for delivering a desired fluid. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

[1] A. Zaher, S. Li, O. Yassine, N. Khashab, N. Pirmoradi, L. Lin, J. Kosel: “Osmotically driven drug delivery through remote-controlled magnetic nanocomposite membranes”. Biomicrofluidics, vol. 9, no. 5, p. 054113, 2015.
[2] P. Song, D. J. H. Tng, R. Hu, G. Lin, E. Meng, and K. T. Yong, “An electrochemically actuated MEMS device for individualized drug delivery: an in vitro study,” Advanced healthcare materials, vol. 2, no. 8, pp. 1170-1178, 2013.
[3] Y. Yi, A. Zaher, O. Yassine, J. Kosel, I.G. Foulds, “A remotely operated drug delivery system with an electrolytic pump and a thermoresponsive valve,” Biomicrofluidics, vol. 9, no. 5, p. 052608, 2015.
[4] K. Moussi and J. Kosel, “3-D Printed Biocompatible Micro-Bellows Membranes,” J. Microelectromech. Syst., vol. 27, no. 3, pp. 472-478, 2018.

1. A miniature delivery system comprising:
a base;
a pumping mechanism attached to the base; and
a housing having needles, the housing being attached to the base so that the pumping mechanism is enclosed by the housing,
wherein the needles are configured to not buckle or break when pressed directly into a skin or organ of a human to which the miniature delivery system is attached to, and
wherein the pumping mechanism is configured to pump a fluid from the housing into the skin or organ, through the needles.

2. The system of claim 1, wherein the pumping mechanism comprises:
electrodes formed on the base;
a receiving coil electrically connected to the electrodes; and
a bellows membrane fixedly attached with one side to the base.

3. The system of claim 2, wherein the bellows membrane and the substrate define a first internal chamber configured to hold water.

4. The system of claim 3, wherein the housing and the bellows membrane define a second internal chamber configured to hold the fluid.

5. The system of claim 4, wherein the bellows membrane is directly attached to the base.

6. The system of claim 4, wherein the bellows membrane is not contacting the housing when in a retracted state.

7. The system of claim 4, wherein the bellows membrane contacts the housing when in an extended state.

8. The system of claim 1, wherein the needles have an inner diameter of 30 to 120 μm.

9. The system of claim 8, wherein the needles have a height between 50 and 1000 μm.

10. The system of claim 1, wherein the pumping mechanism is configured to receive electrical energy in a wireless manner.

11. The system of claim 1, wherein the needles are integrally made with the housing from the same material.

12. A miniature delivery kit for delivering a drug, the kit comprising:
a delivery system; and
means for attaching the delivery system to a skin or organ,
wherein the delivery system includes,
a base,
a bellows membrane directly attached to the base, and
a housing having needles, the housing being attached to the base so that the bellows membrane is enclosed by the housing,
wherein the bellows membrane moves from a retracted state, in which an external face is farthest from an internal face of the housing, to an extended state, in which the external face is closest to the internal face of the housing, and
wherein the external face of the bellow membrane is substantially parallel to the internal face of the housing in both the retracted state and the extended state.

13. The kit of claim 12, wherein the needles are configured to not buckle or break when pressed directly into a skin or organ of a human to which the miniature delivery system is attached to.

14. The kit of claim 12, wherein the means for attaching is a tape.

15. The kit of claim 14, wherein the tape is placed directly over the base.

16. The kit of claim 12, further comprising:
interdigitated electrodes formed on the substrate; and
a receiver coil electrically connected to the interdigitated electrodes.

17. The kit of claim 16, further comprising:
a transmitter coil and a power supply configured to induce electrical energy into the receiver coil and generate electrolysis in water stored in a first chamber defined by the base and the bellow membrane, to actuate the bellows membrane from the retracted state to the extended state.

18. The kit of claim 17, wherein the drug is stored in a second internal chamber, defined by the external face of the bellows membrane and the internal face of the housing, and when the bellows membrane is actuated from the retracted state to the extended state, the drug is delivered through the needles to the skin or organ.

19. A method for delivering a drug to a skin or organ of a human, the method comprising:
loading a delivery system with the drug;
attaching the delivery system directly to the skin or organ by pushing one or more needles directly into the skin or organ, wherein the one or more needles are part of a housing of the delivery system;
actuating a bellows membrane of the delivery system to move from a retracted state to an extended state;
delivering the drug through the one or more needles to the skin or organ; and
removing a power supply from the delivery system to stop the drug delivery.

20. The method of claim 19, further comprising:
moving the bellows membrane only from the retracted state to the extended state.
